SEQUENCE LISTING (4 sequences)

SEQ ID NO: 1 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 2 - protein, 165 aa, Homo sapiens [sequence omitted]

SEQ ID NO: 3 - protein, 20 aa, Homo Sapiens
CKKSIENAKQDLLQLKNVIS

SEQ ID NO: 4 - protein, 920 aa, Homo Sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPPQVPVKRKYLFTFQGEKIESLRSSLQEARSFEEEMEGDPPADYDDRIIATLKAVQDSKLDQVLVEFTCKNQPKPSLPTEWALCGEREDRLELLKLSTFALIITPGDPRLVISSGCATRLFEALEVGAVPVVLGEQVQLPYQDMLQWNEAALVVPKPRVTEVHFLLRSLSDSDLLAMIRRQGRFLWETYFSTADSIFNTVLAMIRTRIQIPAAPIREEAAAEIPHRSGKAAGTDPNMADNGDLDLGPVETEPPYASPRYLRNFTLTVTDFYRSWNCAPGPFHLFPHTPFDPVLPSEAKFLGSGTGFRPIGGGAGGSGKEFQAALGGNVPREQFTVVMLTYEREEVLMNSLERLNGLPYLNKVVVVWNSPKLPSEDLLWPDIGVPIMVVRTEKNSLNNRFLPWNEIETEAILSIDDDAHLRHDEIMFGFRVWREARDRIVGFPGRYHAWDIPHQSWLYNSNYSCELSMVLTGAAFFHKYYAYLYSYVMPQAIRDMVDEYINCEDIAMNFLVSHITRKPPIKVTSRWTFRCPGCPQALSHDDSHFHERHKCINFFVKVYGYMPLLYTQFRVDSVLEKTRLPHDKTKCFKFI

(19) United States
(12) Patent Application Publication
(10) Pub. No.: US 2019/0328839 A1
(43) Pub. Date: Oct. 31, 2019

(54) Treatment of Type 1 Diabetes and Other Conditions Using the Gut Microbiome

(71) Applicant: Claresa Levetan, Bryn Mawr, PA (US)

(72) Inventor: Claresa Levetan, Bryn Mawr, PA (US)

(21) Appl. No.: 16/291,207

(22) Filed: Mar. 4, 2019

Related U.S. Application Data
(63) Continuation-in-part of application No. 15/815,318, filed on Nov. 16, 2017.
(60) Provisional application No. 62/793,989, filed on Jan. 18, 2019.

(51) Int. Cl. A61K 38/21; A61K 9/00; A61P 3/10

(52) U.S. Cl. CPC A61K 38/212; A61P 3/10; A61K 9/0053

(57) ABSTRACT

The disclosure provides new methods for treatment of inflammatory and autoimmune diseases including diseases of impaired glucose homeostasis and includes the usage oral usage immunotherapies that are T lymphocyte helper cell 17 (Th17)-inhibitors and usage is based on the patient's gut microbiome to determine if they are at risk for targeted organ(s) attack by Th17. The delivery of Th17-inhibitors is delivered preferably orally because Th17 lines the mucosa of the gastrointestinal tract. With the treatment of Type 1 diabetes, both a Th17-inhibitor is used with existing or new islet neogenesis agents presented within.

### CROSS-REFERENCE TO RELATED APPLICATIONS

The present application claims priority to U.S. Provisional Application Ser. No. 62/793,989 filed Jan. 18, 2019 and is also a Continuation-in-Part (CIP) of U.S. patent application Ser. No. 15/815,318 filed Nov. 16, 2017, the disclosure of both applications are incorporated herein by this reference.

### REFERENCE TO SEQUENCE LISTING SUBMITTED IN COMPUTER READABLE FORM

The present application contains a Sequence Listing which has been submitted in ASCII format by way of EFS-Web and is hereby incorporated by reference herein in its entirety. The ASCII file was created on Feb. 17, 2019 and named CLEV019_SEQ_ST25, which is 12 kb in size, and which is identical to the paper copy filed with this application.

### FIELD OF THE INVENTION

Embodiments of the present invention provide novel therapies, pharmaceutical compositions, formulations and methods for treatment of impaired glucose homeostasis with treatment for protection from ongoing attack and generation of new islets without the need for transplants.

### BACKGROUND OF THE INVENTION

Diabetes is a worldwide epidemic with no therapies addressing the underlying cause of disease: too few insulin-producing cells. As reported in 2018, women who develop Type 1 diabetes before the age of 10 years, will die, 18 years earlier than their diabetes-free counterparts, and men with early onset Type 1 diabetes will die 14 years earlier (Rawshani et al., 2018 Lancet. 392(10146); 477-486). The largest percentage of patients with Type 1 diabetes who die under the age of 50, do so from diabetic coma and diabetic ketoacidosis (Livingstone et al., JAMA. 2015; 313(1):37-44).

Diabetes remains the leading cause of blindness, amputations and kidney failure necessitating dialysis or kidney transplant. In addition, the annual costs in the US for care of Type 1 diabetes patients are estimated at $14.4 Billion with $429 Billion spent in the US over the lifetime of care for the cohort of patients with Type 1 diabetes (Tao et al., PLoS ONE 5(7): e11501).

New to the field is that in humans, Type 1 diabetes is not only a disease of autoimmunity, but also a disease in which there is lack of regeneration of insulin-producing cells, even in an immune-muted milieu. FIG. 1 was published from the proceedings of an International Summit on Insulin Independence, with participation by world leaders including Dr. Desmond Schatz, 2016 President of the American Diabetes Association (Levetan et al. Diabetes Metab Res Rev. 2013; 29(8):604-6). FIG. 1 presents the broad aspects of the current disclosure.

The Diabetes Control and Complication Trial (DCCT) was the largest randomized trial conducted among Type 1 diabetes patients and was designed by the greatest minds in the field of diabetes and recruited patients from 1983-1989. The study treated half the patients with intensive insulin therapy via injections or insulin pumps to insulin to mimic the pancreas' secretion of insulin with the goal of achieving a Hemoglobin A1C (A1C) within the normal range of less than 6.05%.

The intensively treated arm only achieved a reduction of the AIC down to a level of 7.2% from a baseline of 8.6%, only reaching a 50% diminution in AIC from that hypothesized. At the time that the DCCT study was designed, the importance of all islet hormones were not well understood, and the islet ghrelin and amylin had not been discovered. The integrated role of glucagon, somatostatin, pancreatic polypeptide and islet ghrelin had not been elucidated. The beta cell hormone, amylin, which is co-secreted with insulin in equimolar concentrations, restrains glucagon and reduces glycemic excursions and reduces premeal insulin requirements, was not discovered until 1988 (Levetan et al., Diabetes Care. 2003 January; 26(1):1-8).

FIG. 2 demonstrates why complications occur in patients with Type 1 diabetes. Insulin is the only treatment now available for Type 1 diabetes. Insulin is only one of 6 hormones necessary to work in a highly integrative system within the Islets of Langerhans (islets). Without wholly functional islets, normal glucose homeostasis does not occur. See FIG. 2.

Insulin independence and the ability to achieve normal glucose levels for both Type 1 and 2 diabetes will rely on the generation of new islets, not just the production of new insulin-producing beta cells. The importance of a fully functional islet within the pancreas is one of two keys to insulin independence for patients with Type 1 diabetes. The second being pancreatic protection from autoimmune destruction.

Studies have confirmed that the REG gene is the triggering factor for new islet formation from extra-islet progenitor cells within the pancreatic ductal tissue, rather than from existing beta cells within islets (Kapur et al., Islets. 2012; 4(1):40-8, Li et al., Peptides. 2009; 30(12):2242-9, Levetan. J Diabetes. 2010; 2(2):76-84). More than 200 peer-reviewed articles have been published on the role of the REG gene in islet neogenesis both during and after fetal development (Li Q., Xiong X., Liu J L. (2014) In: Islam M. Islets of Langerhans, 2. ed. Pages 1-40. Springer, Dordrecht, Levetan. J Diabetes. 2010; 2(2):76-84). The REG gene is expressed during acute injury in the pancreas in order to generate new islets for survival of the individual.

The work of the late Dr. Lois Jovanovic demonstrates ten consecutive pregnant Type 1 patients in her clinic, with a mean duration of Type 1 diabetes for 21 years, who had no detectable baseline C-peptide, a marker of endogenous insulin production from the pancreas, prior to pregnancy. There was a rise within all patients of C-peptide levels into the normal range. See FIG. 3 for a graph of the aforementioned results.

Islet neogenesis is known to occur in pregnancy due to upregulation of the REG gene and accompanied by decreased immunity in the mother so that the fetus is not rejected as “foreign” by the mother. Upregulation of the REG gene is seen in pregnancy in other mammalian species (mouse Genomics Informatics. Mus musculus 11 days pregnant adult female ovary and uterus cDNA, RIKEN full-length enriched library, clone:5033401N17 product: regenerating islet-derived 1, full insert sequence. http://www.ebi.ac.uk/cgi-bin/emblfetch?AK133506).

Despite the body's intrinsic ability to regenerate new cells when an organ is injured or attacked by autoimmunity, as in the case of Type 1 diabetes, the balance between destruction and regeneration will determine if the organ system survives. The REG gene has been shown by many teams to be upregulated in times of acute injury of the pancreas in order to survive. With the use of exogenous insulin, patients with Type 1 diabetes live, but they are not spared from serious complications and shortened lifespans.

The entire islet structure with the beta cell intricately positioned within the islet is required for normal glucose homeostasis. This inventor has spent the last two decades attempting to answer why successful therapies that reverse diabetes in animal models of Type 1 diabetes (NOD mice) have not been translated into man.

By distinguishing between the differences between islets in mice versus humans, there is a vast distinction between cell types, distribution and percentages of cell types. The human islets make six hormones that are all necessary for glucose homeostasis (Levetan et al., Endocr Pract. 2013 March-April; 19(2):301-12). The photomicrograph in FIG. 4 demonstrates that within the human islets, the insulin-producing beta cells, stained in red, are physically connected with 70% of the four other cell types within the islet including the glucagon-secreting alpha cells in green, and the somatostatin-producing delta cells in blue. The large irregularly-shaped black holes within the human islet are blood vessels that are lined with smooth muscle and are innervated by the nervous system. Islets comprise 2% of the pancreatic mass but receive 20% of the blood flow within the pancreas.

All six of the human islet hormones, made in the five cell types within the islet play an important role in maintaining glucose levels within the normal range (Levetan et al., Endocr Pract. 2013 March-April; 19(2):301-12). In man, there are much lower percentages of beta cells with higher percentages of other cell types compared to mice (Cabrera et al., Proc Natl Acad Sci USA. 2006; 103:2334-2339).

While the scientific community and peer-reviewed journals still use the term beta-islets, beta cells and islets are not synonymous. This inventor has published on genomic differences between islet neogenesis from progenitor cells found within the extra-islet pancreatic ductal tissue and beta cell regeneration from existing beta cells within islets (Levetan., J Diabetes. 2010 June; 2(2):76-84).

This inventor has studied the neural connections between the human islet and the central and peripheral nervous systems. As shown in FIG. 5, there is a very complex system both between different cell types within the islets and the innervation of the large blood vessels within the human islet that are lined with smooth muscle innervated by the nervous system. The diagram in FIG. 5 demonstrates the complex connection between the Islets of Langerhans to both the brain and autonomic nervous system and illustrates the role of the six islet hormones including the islet hormone, islet ghrelin, which has now shown to play a role in glucose homeostasis. Only with a completely functioning 5-celled islet making six hormones, is there healthy glucose homeostasis (Barreto et al., Am J Physiol Gastrointest Liver Physiol. 2010; 299(1):G10-22).

Autopsy studies among both Type 1 and Type 2 diabetes patients demonstrate that there are very limited remaining insulin-producing beta cells within islets, and that islet structures are also greatly diminished in both Type 1 and 2 diabetes (Doniach and Morgan. 1973 Clin Endocrinol (Oxf). 2(3):233-48, Deng et al., 2004. Diabetes. 53(3):624-32). These studies illustrate that the insulin and amylin secreting beta cells are integral to the survival of the entire islet in both Type 1 and 2 diabetes.

This inventor has been issued patents for 7-15 amino-acid peptides located within Reg gene proteins and optimized Reg peptides for islet neogenesis in both patients with Type 1 and 2 diabetes (U.S. Pat. Nos. 9,511,110, 9,321,812, 8,785,400, 7,393,919, 7,714,103, 7,989,415, 8,383,578, 8,211,430, 10016482, 10010580, 10010579, 10010578, and 10010577). Reg peptides have demonstrated the ability to transform human pancreatic ductal tissue (tissue without islets or beta cells) into new islets (Li et al., Peptides. 2009 December; 30(12):2242-9, Levetan et al., Endocr Pract. 2008 December; 14(9): 1075-83).

Gold standard BrdU studies distinguishing whether new insulin-producing cells are derived from existing beta cells or extra-islet pancreatic ductal tissue, specifically found that the 14- and 15-amino acid Reg gene peptides which have been used in humans with Type 1 and 2 diabetes patients, resulted in new islets from extra-islet ductal tissue rather from existing beta cells (Kapur et al., Islets. 2012; 4(1):40-8). These 14- and 15-amino acid Reg peptides were also shown to increase pancreatic islet progenitor markers Ngn3, Nkx6.1, Sox9 and Ins (Kapur et al., Islets. 2012; 4(1):40-8). The newest small islets formed directly from extra-islet ductal cells within the pancreas, stain positive for the Reg gene peptide (Guo et al., Diabetes. 2010; 59(suppl 1).

FIG. 6 shows the representative peptide the structures of the two Reg peptides studied above. The circled area in red represents the amino acid regions within the protein that bind to the REG receptor, EXTL3. The peptides within the circled area of the location of the 14 and 15-amino acid peptides that have demonstrated in vitro and in vivo to generate new islets after binding to the REG gene receptor, EXTL3 that is found in pancreatic ductal tissue (Levetan et al., Endocr Pract. 2008 December; 14(9): 1075-83).

This inventor has identified the 20-amino acid binding region within the 920 amino-acid REG gene receptor, EXTL3, and in this invention presents this binding region from which small molecules, stimulatory antibodies or receptor analogs may be generated for islet neogenesis. Of note is the specificity of the Reg peptides to the pancreatic ductal tissue and not to other organs. FIG. 7 is a fluorescein isothiocyanate-labeled (FITC-labeled) staining study demonstrating that an injection of labeled Reg peptide goes to ductal tissue and not to other organs (Pittinger et al., Pancreas. 2007; 34(1):103-11). FIG. 7 is a comparative image that show that there was significant staining in the pancreatic ducts with a FITC-tagged 15-amino Reg peptide when injected intraperitoneally (A), but no staining in other organs examined (e.g. kidney) (B).

Human trials have been conducted with Reg peptides among Type 1 and Type 2 diabetes patients demonstrating safety and potential for efficacy. A human trial among Type 1 diabetes patients with the disease for 20 years demonstrated a 27% (p=0.0058) rise in endogenous insulin within eight weeks of daily therapy among patients with Type 1 diabetes for 20 years (Ratner et al., 2005. https://professional.diabetes.org/abstract/double-blind-placebo-controlled-trial-islet-neogenesis-gene-associated-protein-ingap-Type-1).

This trial also demonstrated that GAD antibodies were significantly higher in an active treatment arm, which likely indicates that the presence of new insulin-producing beta cells triggered the autoimmune process. The biggest side effect in all of the Type 1 and 2 studies with Reg peptides in man have been injection site pain, soreness and swelling (Dungan et al., Diabetes Metab Res Rev. 2009 September; 25(6):558-65, Watkins et al., (https://ada.scientificposters.com/epsAbstractADA.cfin?id=1).

A study of five sets of identical twins also demonstrated that when one twin developed Type 1 diabetes and received a transplant of the pancreatic tail from the sibling without diabetes, there was a rapid autoimmune attack on the transplanted pancreas, with insulin dependence rapidly returning in all the twins with Type 1 diabetes (Pugliese et al., Diabetes Manag (Lond). 2011; 1(2): 229-238). The human trials with Reg peptide among Type 1 patients demonstrated that GAD antibodies were significantly higher in an active treatment arm, and the study of pancreas transplants among identical twins demonstrate the need among Type 1 diabetes patients for both the usage of an immune therapy to prevent ongoing attack in the face of the body's generation of new insulin-producing cells and an islet neogenesis agent.

More than 300 studies demonstrating success in reversing diabetes in Type 1 diabetes mouse models (NOD mice) have not been successful in man (Matthews et al., Clin Exp Immunol. 2010; 160(2): 176-184). Even when combinations of immune therapies have been given in humans with Type 1 diabetes, there has not been regeneration of insulin producing cells or insulin independence, thus Type 1 diabetes in man has been defined as both a disease of autoimmunity and lack of regeneration of insulin-producing cells even in an immune muted milieu.

### SUMMARY OF THE INVENTION

The present invention relates to novel therapies, pharmaceutical compositions and methods for treating conditions, which are autoimmune or inflammatory including Type 1 diabetes based on the individual demonstrating an aberrant gut microbiome. This inventor provides new compositions for Type 1 diabetes using both an oral T helper lymphocyte 17 (Th17)-inhibitor with an islet neogenesis agent, which may include a Reg peptide or new to the art, are stimulatory antibodies, small molecules and receptor binding site analogs generated by this inventor from the identification of the 20 amino-acid binding site on the 920 amino-acid REG gene receptor.

This invention also includes using an individual's gut biome for the diagnoses of autoimmune and inflammatory diseases, including Type 1 diabetes that result from a Th17-initiated autoimmune or inflammatory attack to an organ system based upon the individual's gut microbiome. The gut microbiome forms the body's immune barrier through its impact on the Th17, which is now considered the master immune regulator. Th17 lines all the surfaces of the gut including the mouth, tongue, throat, stomach, large and small intestines. Until recently, it was thought that Th17 was only located in pockets of lymphoid tissue in the gastrointestinal tract known as Peyer's patches. It is now known that Th17 is present along the entire gastrointestinal tract. Because 70% of the body's immune system is located within the gastrointestinal tract, the relationship between the gut microbiome and the formation of the immune system is now better understood and the role of the gut microbiome impact on Th17's formation of immunity has recently been better understood (Vighi et al., Clin Exp Immunol. 2008; 153(Suppl 1): 3-6, Ivanov et al., Cell Host Microbe. 2008:16; 4(4): 337-349, Omenetti and Pizarro. 2015: Front. Immunol. 6:639, Kumar et al., 2016: Immunity 44, 659-671, Hirota et al., Nat Immunol. 2013; 14(4): 372-379).

A healthy gut microbiome specifically teaches Th17 to recognize the ecosystem of microorganisms in the normal gut microbiome as “self” and creates an immune memory not to attack the body's own organ systems, when Th17, considered the master immune regulator and is triggered to begin an immune attack against an outside antigen, such as with an infection. It is the gut microbiome that forms the body's immune barrier through its interaction with Th17 (Ivanov et al., Cell Host Microbe. 2008:16; 4(4): 337-349, Omenetti and Pizarro. 2015: Front. Immunol. 6:639, Kumar et al., 2016: Immunity 44, 659-671, Hirota et al., Nat Immunol. 2013; 14(4): 372-379).

In humans, the gut microbiome is established within the first few years of life, by which time, the immune system has developed an immune barrier that is based upon the gut microbiome presented to Th17. The normal immune barrier occurs when the normal gut microbiome trains Th17 to recognize the normal gut microbiome as “self.” When the gut biome does not have the complete amounts and concentrations of microorganisms found in healthy gut microbiomes, Th17 does not have all necessary microorganisms to properly imprint upon the immune system. Diseases and disease combinations can be identified by their unique gut microbiome profile and compared to populations with normal, healthy gut microbiomes.

For example, among patients with Type 1 diabetes, there are reduced numbers of lactobacillus, bifidobacterium, akkermansia, roseburia faecis, faecalibacterium prausnitzii and bacteroides (Gianchecchi and Fierabracci 2019 Int. J. Mol. Sci. 2019 20, 283, Zheng et al, Diabetes Metab Res Rev 2018; 34:e3043, Han et al., Int J Mol Sci. 2018; 19(4): 995). Without the normal amounts and concentrations of these specific microorganisms, there is a specific deficiency in the microorganisms presented to Th17. An incomplete library of microorganisms is unable to generate a normal immunity that reads the insulin-producing cells of the pancreas as “self.” When there are lower than normal quantities and ratios of gut microorganisms, the immune system will not form specific recognition of organ systems for the specific diseases identified by the unique deficiencies in the gut microbiome. Aberrant gut microbiomes specifically reflected by different diseases and combination of diseases create a weakened immune system, and these diseases states are reflected in Th17 lacking the ability to recognize organ(s), as “self.”

The weakness in the body's immune system for those with Type 1 diabetes is caused by an insufficient quantity and concentration of specific microorganisms. The gut microbiome is a library of antigens that serves as an identity manual as to what is “self” and thereby prevents the immune system from reacting against the body itself, including, for example, reacting against insulin-producing cells of the pancreas in Type 1 diabetes.

When Th17 is not presented with the full complement of normal microorganisms, the immune barrier is incompletely formed and lacks the ability to recognize its own organ systems as “self” and may attack an organ system(s) based upon the individual's aberrant gut microbiome. When an outside antigen (i.e., as during an infection) attacks an individual with a normal gut microbiome, Th17 is triggered to initiate a downstream targeted reaction only to the outside antigen (the infection). However, in a patient with a Type 1 diabetes microbiome, the immune system may see the insulin-producing cells as foreign and attack the pancreas leading to an acute onset of Type 1 diabetes.

An individual with an aberrant gut biome lacks the mechanism that prevents the activation of Th17 cells that would normally not react to self-tissues or organs. This occurs because a normal gut biome acts to “teach” the Th17 cells what is “self” during the antigen presentation phase of the immune response and leads to clonal anergy or deletion of any immune cells that would react against self. In an individual with a gut biome that is deficient, the Th17 cells remain potentially active against one's own organ system(s), such that when an external antigen is presented, the immune system will react both against the foreign antigen, and unfortunately, any other tissue or organ that “looks like” the foreign antigen. This may be the critical aspect of the etiology of autoimmune diseases like Type 1 diabetes.

In the specific instance of Type 1 diabetes, the aberrant gut biome of such individuals destined to suffer from Type 1 diabetes lack the ability to “teach” the Th17 cells what is self. This leads to an immune cascade against a later presented foreign antigen and that immune response is directed to not only the foreign antigen, but also to other “self” tissue or organ that “looks like” the foreign antigen to the immune system. In this instance, Th17 mistakenly specifically attack the beta cells of the pancreas and the individual becomes symptomatic of Type 1 diabetes. Diseases such a Type 1 diabetes, other autoimmune diseases, and disease combinations with abnormal gut microbiomes are at risk for a Th17-initiated attack, and therefore would benefit from usage of a Th17-inhibitor. The islet neogenesis agents presented may also be used in Type 2 diabetes, PreDiabetes and latent autoimmune diabetes of adulthood (LADA).

In particular, the disclosure provides for a method of treating or preventing impaired glucose homeostasis such as Type 1 and 2 diabetes and latent autoimmune diabetes of adulthood (LADA) in a subject. The method involves the steps of: mapping the subject's gut microbiome; comparing the subject's gut microbiome results with known mapped gut microbiomes of individuals suffering from particular forms of impaired glucose homeostasis; determining the particular type of glucose impairment of the subject based on the subject's mapped gut microbiome matching a known mapped gut microbiome of an individual suffering from a particular form of impaired glucose homeostasis; using one or more islet or beta cell regeneration or replacement therapies; and administering a T helper lymphocyte 17 (Th17)-inhibitor at an amount that is effective for protection of beta cells from immune-mediated destruction in the subject.

In an additional embodiment, the disclosure provides for a method of method of treating or preventing one or more autoimmune diseases or conditions in a subject. This method involve the steps of: mapping the subject's gut microbiome; comparing the subject's gut microbiome results with known mapped gut microbiomes of other individuals suffering from a particular forms autoimmune diseases or conditions; determining the particular type of autoimmune disease or condition of the subject based on the subject's mapped gut microbiome; matching a known mapped gut microbiome of individuals suffering from a particular form of autoimmune disease or condition; and administering a Th17-inhibitor at an amount that is effective for alleviating or preventing symptoms of the autoimmune disease or condition in the subject.

Finally, the disclosure provides for a method of diagnosing and treating one or more diseases that is based on a Th17-initiated inflammatory or autoimmune attack in a subject. The method involves the steps of mapping the subject's gut microbiome; comparing the subject's gut microbiome results with known mapped gut microbiomes relating to aberrant gut microbiomes of individual's suffering symptoms of Th17-initiated inflammatory or autoimmune attacks; and diagnosing the subject's particular Type disease based on the subject's mapped gut microbiome matching a known mapped gut microbiome of an individual suffering from a particular form of Th17-initiated inflammatory or autoimmune attack. The method continues by administering a Th17-inhibitor at an amount that is effective for alleviating or preventing symptoms of the Th17-initiated inflammatory or autoimmune attack in the subject.

### BRIEF DESCRIPTION OF THE SEQUENCES

SEQ ID NO: 1 is the amino acid sequence of Interferon alfa-2a.

SEQ ID NO:2 is the amino acid sequence of Amino Acid Sequence of PEGylated Interferon alfa-2a (PEGylated with a branched 40 kDa PEG chain).

SEQ ID NO:3 is the 20-amino acid binding region sequence found within the human Reg gene protein receptor, EXTL3.

SEQ ID NO:4 is the 920-amino acid human Reg protein receptor, also known as EXTL3.

### BRIEF DESCRIPTION OF THE DRAWINGS

The accompanying drawings illustrate certain aspects of embodiments of the present invention and should not be used to limit the invention. Together with the written description the drawings serve to explain certain principles of the invention.

Additionally, the patent application file contains at least one drawing executed in color. Copies of this patent or patent application publication with color drawing(s) will be provided by the Office upon request and payment of the necessary fee.

FIG. 1 is a schematic diagram of the overall concept if the present disclosure that in man, it is now accepted by thought leaders in the field of Type 1 diabetes, that Type 1 Diabetes in man is a disease of both autoimmunity and lack of regeneration of insulin-producing cells, even when one or more immunosuppressive therapies are used.

FIG. 2 is a graph demonstrating glucose fluctuations with insulin vs normal pancreas function.

FIG. 3 is a bar graph demonstrating a C-peptide/Endogenous insulin concentration before pregnancy and at 10 weeks of gestation among patients with a 21-year history of Type 1 diabetes.

FIG. 4 is a photomicrograph of a human islet and mouse islet.

FIG. 5 is a diagram demonstrating the complex connection between the Islets of Langerhans to both the brain and autonomic nervous system and illustrates the role of the six islet hormones including the islet hormone, islet ghrelin, which has recently been shown to play a role in glucose homeostasis.

FIG. 6 demonstrates the representative peptide the structures of two Reg peptides that have been used in man.

FIG. 7 is a comparative photomicrograph indicating that there was significant staining in the pancreatic ducts with fluorescein isothiocyanate-labeled (FITC-labeled) with a 15 amino-acid Reg peptide when injected intraperitoneally (A), but no staining in other organs examined (e.g. kidney) (B).

FIG. 8 is SEQ ID NO: 1, the amino acid sequence of Interferon alfa-2a.

FIG. 9 is the representative peptide structure of Interferon alfa-2a.

FIG. 10 is SEQ ID NO:2, the amino acid sequence of PEGylated Interferon alfa-2a.

FIG. 11 is a representation of PEGylated Interferon Alfa 2a and Type 1 Interferon Receptor

FIG. 12 is SEQ ID NO:3, the 20-amino acid peptide binding region for Reg peptides found within the human REG gene protein receptor, EXTL3.

FIG. 13 is SEQ ID NO:4, the 920-amino acid human Reg receptor, also known as EXTL3.

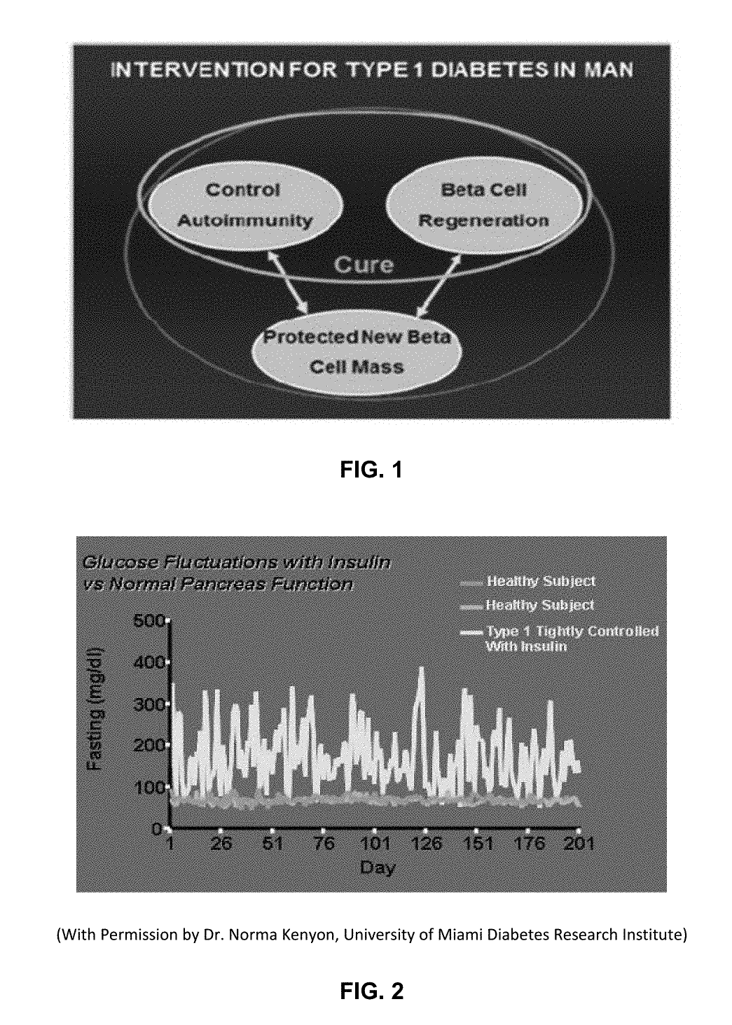

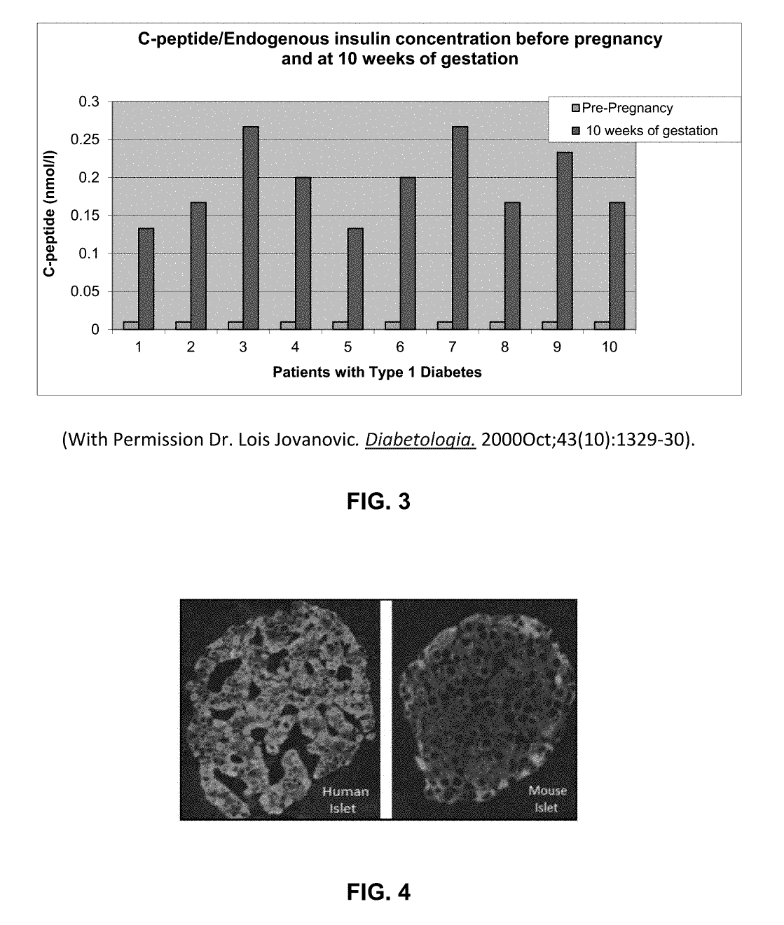

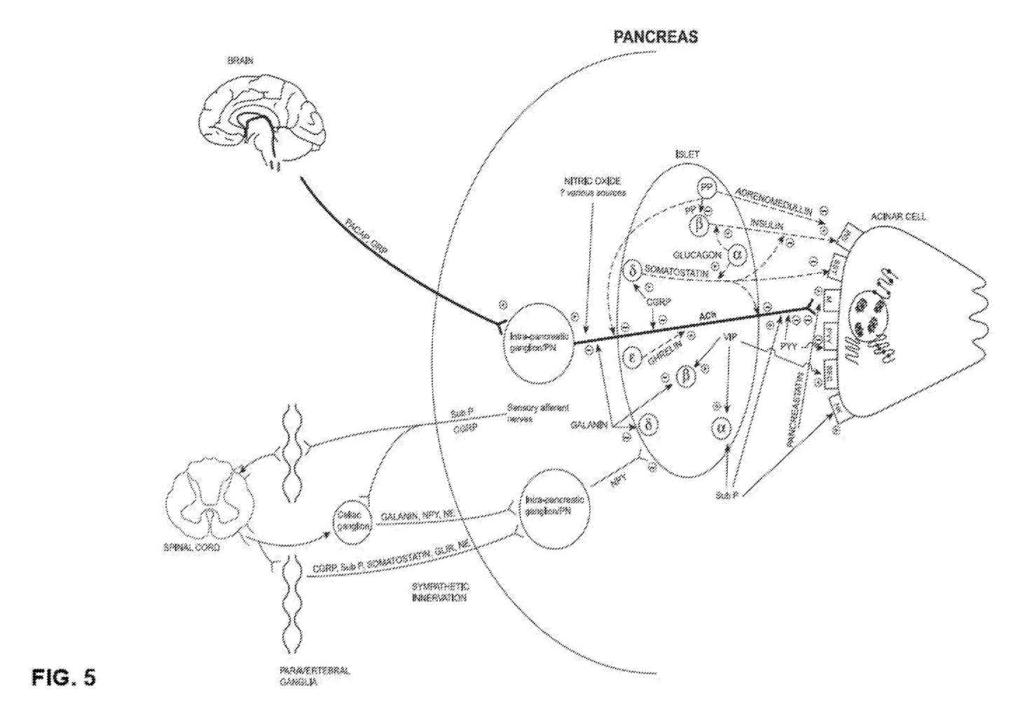

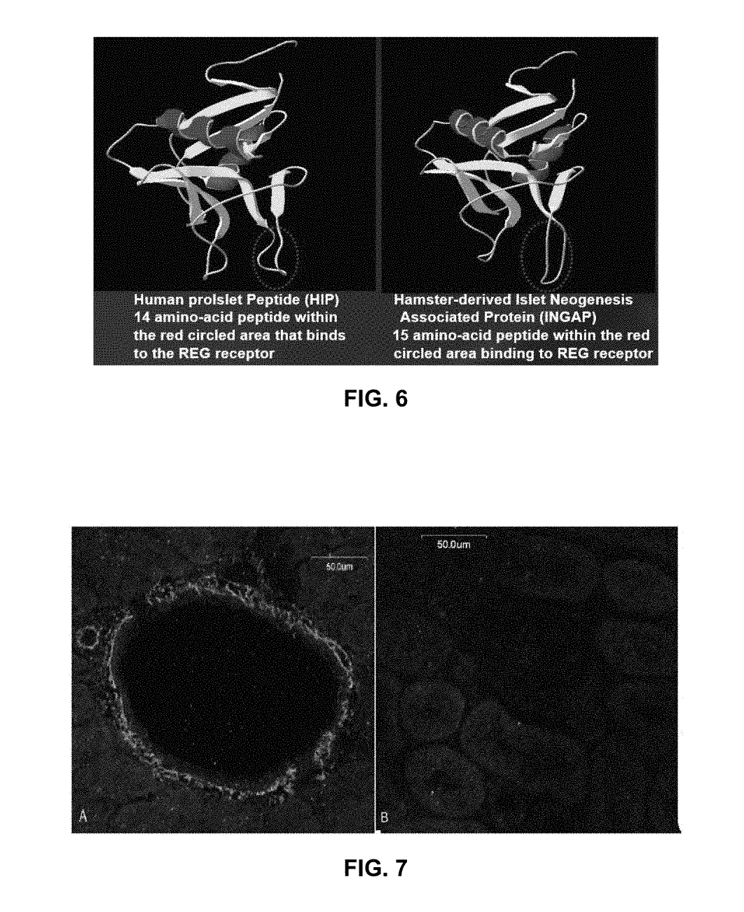

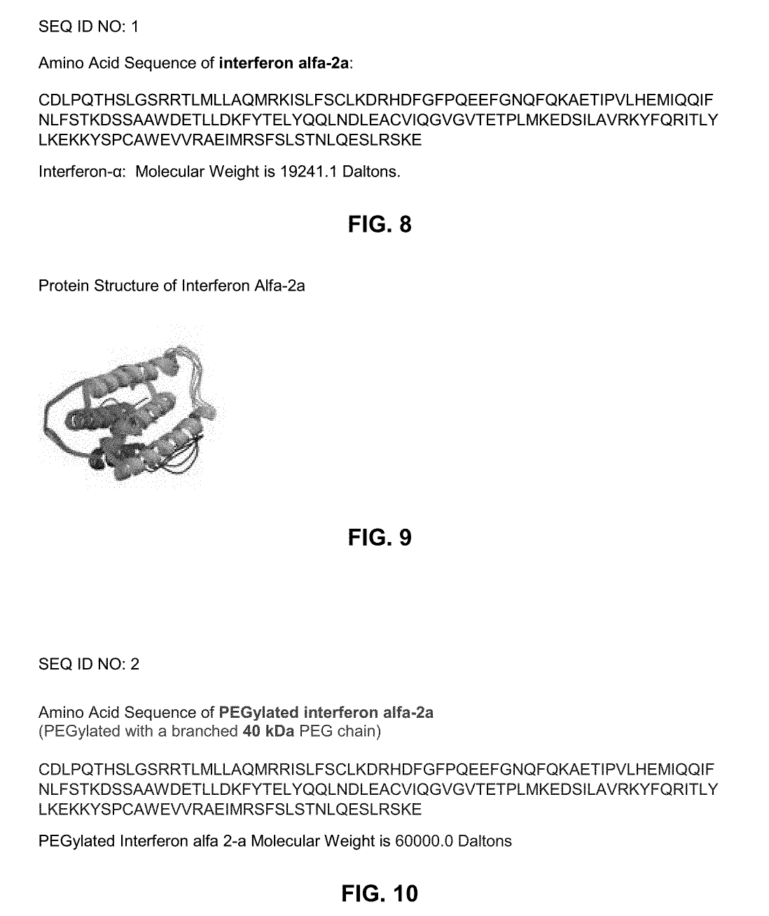

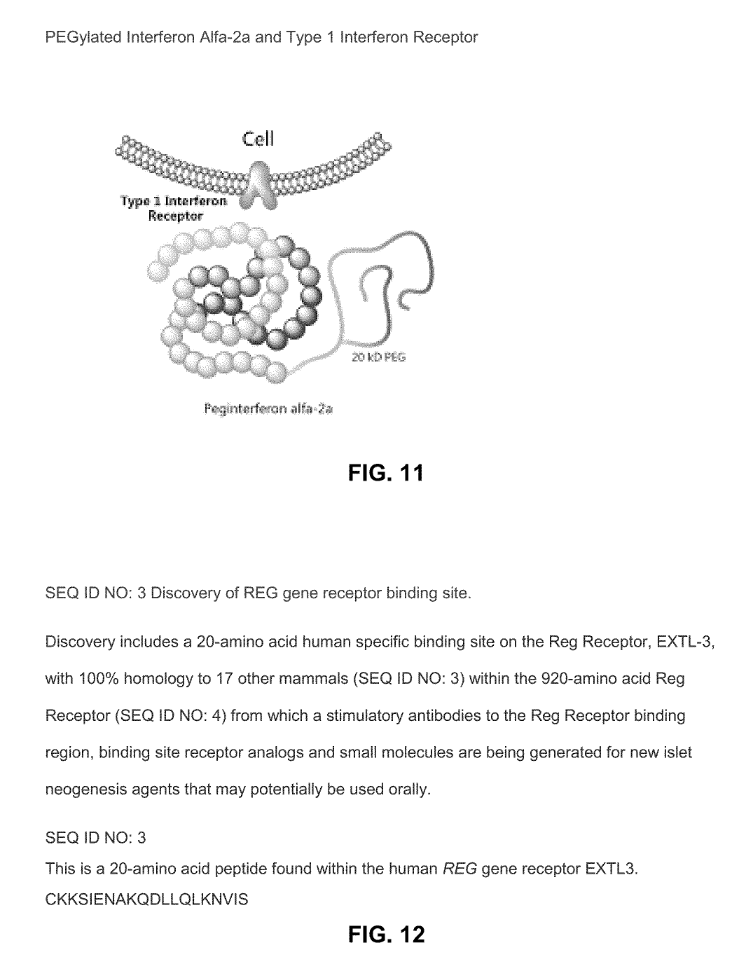

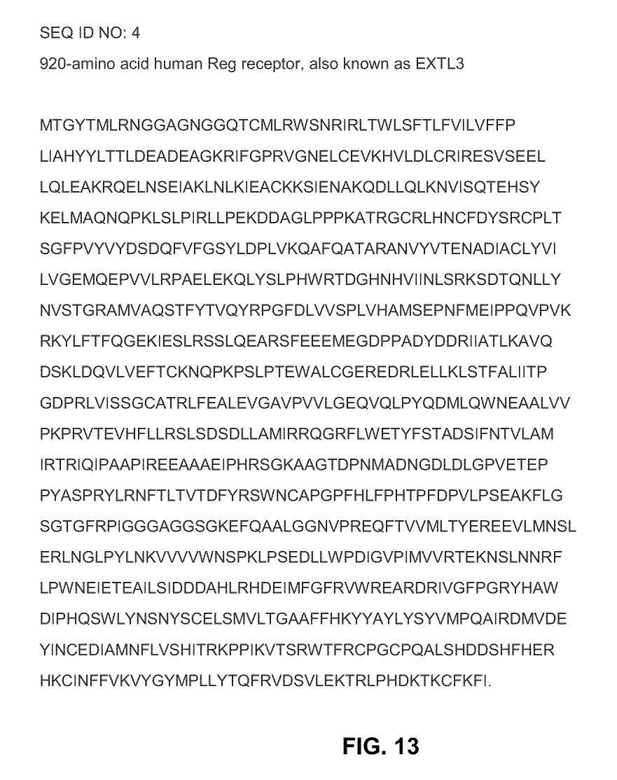

### DEFINITIONS

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the term “about” is understood as within a range of normal tolerance in the art, for example within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of diabetes, diminishment of extent of disease, delay, slowing, or prevention of disease progression, amelioration, palliation or stabilization of the disease state, and other beneficial results described below. Symptoms of Type 1 diabetes include low or inadequate levels of insulin or insulin activity, frequent urination, excessive thirst, extreme hunger, unusual weight loss, increased fatigue, irritability, blurry vision, genital itching, odd aches and pains, dry mouth, dry or itchy skin, impotence, vaginal yeast infections, poor healing of cuts and scrapes, excessive or unusual infections, hyperglycemia, loss of glycemic control, fluctuations in postprandial blood glucose, fluctuations in blood glucagon, fluctuations in blood triglycerides. Diabetes may be diagnosed by methods well known to one of ordinary skill in the art. For example, commonly, diabetics have a plasma blood glucose result of greater than 126 mg/dL of glucose. Pre-Type 1 diabetes, which may also be treated by the compositions and methods of the invention is commonly diagnosed by autoimmune antibodies (GAD65, insulin, IA-2 and ZnT8) found in the blood of family members who have Type 1 diabetes.

As used herein, “reduction” of a symptom or symptoms (and grammatical equivalents of this phrase) means decreasing of the severity or frequency of the symptom(s), or elimination of the symptom(s).

As used herein, “impaired glucose homeostasis” is a diminished capacity in a subject for regulating glucose by a system of feedback controls, so as to stabilize health and functioning. Conditions that are associated with or are a risk factor for impaired glucose homeostasis include new onset Type 1, previously existing Type 1 and 2 diabetes with unusual characteristics or poor response to medication that may have positive autoimmune antibodies for diabetes, latent autoimmune diabetes of adulthood (LADA) such as glutamic acid decarboxylase-65 (GAD65), insulin, IA-2 and ZnT8 autoimmunity, and any condition in which a family member of a patient with Type 1 diabetes has GAD65, insulin, IA-2 and ZnT8 antibodies or any antibodies that are markers for the potential of Type 1 diabetes in the future or any forms of diabetes, which does not respond to oral diabetic agents or non-insulin injectables or any case in which a patient is unresponsive to traditional medications including insulin and the usage of oral Interferon alfa-2a is helpful for the patient when given with an islet neogenesis agent, beta regeneration agent, islet, beta or stem cell transplant or device containing islets, stem cells or receipt of an implantable device containing

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “subject” or “patient” or “individual” is a mammal, typically a human, but optionally a mammalian animal of veterinary importance, including but not limited to horses, cattle, sheep, dogs, and cats. “Patient” and “subject” may be used interchangeably herein.

As used herein, a “therapeutically effective amount” of a drug or agent is an amount of a drug or agent that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

As used herein, a “therapeutically effective amount” of a drug may also be an amount of a drug that when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a therapeutically effective amount may be administered in one or more administrations.

As used herein, an “effective amount” of a drug or agent (and grammatical equivalents of this phrase, e.g. “amount of X that is effective”) is an amount of a drug or agent that will have the intended pharmacological or pharmacodynamic effect. The “effective amount” may apply to in vivo, in vitro, or ex vivo applications of the drug or agent.

Interferon alfa-2a is known in the art in its recombinant, native and pegylated forms.

PEGylated Interferon alfa-2a (PEGylated with a branched 40 kDa PEG chain) with synonyms including rHUPEG-IFN-alfa-2a, rHuPEG-IFN-a 2a, rHUPEG-IFN alfa-2a, rHUPEGr-INFalfa-2a, PEGylated Interferon-alfa-2a, PEGylated Interferon alfa-2a, PEGylated Interferon-a 2a, PEGylated Interferon-alfa-2a, and Pegasys has CAS Registry No. 198153-51-4 and has an approximate molecular weight of 60,000 Daltons.

### DETAILED DESCRIPTION OF THE INVENTION

Embodiments of the present invention provide novel therapies, pharmaceutical compositions, formulations and methods for treatment of impairment of glucose homeostasis with treatment for protection from ongoing attack and generation of new islets without the need for transplants. The usage of the immune therapy presented, an oral T helper lymphocyte (Th17)-inhibitor is based upon the patient's gut microbiome and should a patient without Type 1 diabetes demonstrate the specific gut microbiome seen among human patients with Type 1 diabetes, then that individual is at risk for a specifically targeted Th17-initiated autoimmune pancreatic attack.

Usage of an oral Th17-inhibitor is presented, which may be used for inhibition of attack to the pancreas in Type 1 diabetes or potentially used in specific diseases, such as autoimmune related diseases, reflected by abnormal gut microbiomes for which a Th17-inhibitor may treat the disease or prevent the disease in those at risk. In addition to a Th17-inhibitor specific for Type 1 diabetes, the therapy includes an islet neogenesis agent, which may include a Reg peptide or an islet neogenesis agent developed from the 20 amino-acid binding region on the REG gene receptor, presented within, from which stimulatory antibodies to the REG gene binding site receptor, a receptor binding site analog or small molecule to REG gene receptor binding site are generated for the purposes of islet neogenesis. Such treatment with an islet neogenesis agent including the islet neogenesis therapies generated to the 20-amino acid binding region on the REG receptor may also be used for Type 2 diabetes and PreDiabetes. The Th17-inhibitor may also be used with islet or beta cell transplantation.

In efforts to explore new methods of delivery of an islet neogenesis peptide, this inventor explored the human REG gene receptor, EXTL3. New to the art, this inventor identified a 20-amino acid peptide region on the 920-amino acid EXTL3 protein, from which small molecules, stimulatory antibodies or analogs to the receptor are being developed for a therapy for islet neogenesis that can be delivered orally for usage in both Type 1 and 2 diabetes.

Most therapies for Type 1 human patients have posed risks for systemic immunosuppression, cancer, and rare, but fatal viral conditions like Progressive Multifocal Leukoencephalopathy (PML). An ideal therapy would be one that is specific to the pancreas, and does not systemically suppress the immune system, and is safe for both children and adults. In searching for a safe and effective means of preventing autoimmune attack among patients with Type 1 diabetes, the unique patterns within the gut microbiome among patients with Type 1 diabetes were explored and the potential for a therapy that could be used orally was considered by this inventor.

The gut microbial diversity increases from birth to an adult-like microbiome within the first few years of life. Once established, the composition of the gut microbiome is relatively stable throughout adult life but can be altered as a result of antibiotics taken for medical usage or potentially by the ingestion of animals or crops in which antibiotics were used. New science on the gut microbiome demonstrates consistent aberrations in the gut microbiome within specific disease states compared to healthy gut biomes.

Among patients with Type 1 diabetes, there are reduced numbers of lactobacillus, bifidobacterium, akkermansia, roseburia faecis, faecalibacterium prausnitzii and bacteroides (Gianchecchi and Fierabracci 2019 Int. J. Mol. Sci. 2019 20, 283, Zheng et al, Diabetes Metab Res Rev 2018; 34:e3043, Han et al., Int J Mol Sci. 2018; 19(4): 995). In evaluating the impact of the gut microbiome on the immune system, this inventor posits that aberrations in the gut biome lead to a specific attack on organs not seen as “self” based upon the individual's gut microbiome.

The gut microbiome forms the body's immune barrier through its impact on the master immune regulator, T helper lymphocyte 17 (Th17) (Ivanov et al., Cell Host Microbe. 2008:16; 4(4): 337-349, Omenetti and Pizarro. 2015: Front. Immunol. 6:639, Kumar et al., 2016: Immunity 44, 659-671, Hirota et al., Nat Immunol. 2013; 14(4): 372-379). A healthy gut microbiome specifically teaches Th17 to recognize the vast ecosystem of microorganisms in the normal gut biome as “self” and creates an immune memory not to attack the body's own organ systems when Th17 is triggered to begin an immune attack against an outside antigen, such as with an infection.

Only in the last decade have immunologists better understood the role of Th17 in triggering a cascade of inflammation and autoimmunity, now Th17 is being referred to as the master immune regulator. Until recently, it was thought that Th17 was only located in pockets of lymphoid tissue in the gastrointestinal tract known as Peyer's patches. It is now known that Th17 is present along the entire gastrointestinal tract. Seventy percent of the body's immune system is located within the gastrointestinal tract (Vighi et al., 2008 Clinical and Experimental Immunology 153(Suppl. 1) 3-6). New to the art, is that a therapy whose mechanism of action is inhibiting Th17 would be given orally.

The normal immune barrier is complete when the normal gut microbiome trains Th17 to recognize the normal gut microbiome as “self.” When the gut biome does not have the complete normal array of microorganisms found in a healthy person's gut microbiome, Th17 does not have all necessary microorganisms to properly imprint upon the immune system. Without the normal amounts and ratios of these specific microorganisms, there is a deficient profile presented to Th117. An incomplete library of microorganisms is unable to generate a normal immunity that reads aberrant gut microbiomes. For example, Th17 initiates an autoimmune attack against insulin-producing cells based on the immune system formed by Th17's interaction with the gut microbiome for Type 1 diabetes. Diseases and disease combinations can be identified by their unique gut microbiome profile.

This inventor posits that when there are lower than normal amounts and ratios of gut microorganisms, which are unique to different diseases and combination of diseases, the immune system will function abnormally. Aberrant gut microbiomes, specifically reflected by different diseases and combination of diseases, create a weakened immune system, that when triggered by an outside antigen such as an infection, leads to a Th17 immune and inflammatory attack on the external antigens and may read pancreatic insulin-producing cells as foreign and attack the pancreas based on the abnormal gut microbiome that is specific to Type 1 diabetes.

This inventor posits that the gut microbiome is a library of antigens that serves as an identity manual as to what is “self” and thereby prevents the immune system from reacting against the body itself including reacting against insulin-producing cells of the pancreas in Type 1 diabetes, the myelin sheath of neurons in multiple sclerosis (MS), which has its own unique microbiome (Chen et al, 2016 Scientific Reports 6:28484) or peripheral neurons in Amyolateral Sclerosis (ALS), which has its own microbiome (Wright et al, Biol Res Nurs. 2018: 20(5):513-521), or Parkinson's Disease, with its unique gut microbiome (Sampson et al, 2016, Cell 167, 1469-1480) results in attack to the basal ganglia causing motor deficits.

Researchers have long discussed the etiology of Alzheimer's Disease as an inflammatory disease and even have specifically discussed the role of the gut biome in the development of Alzheimer's Disease (Vogt et al., Scientific Reports, 2017 (7) Article 13537, Hu, Sci China Life Sci. 2016; 59(10):1006-1023, https://doi.org/10.1007/s11427-016-5083-9 Jiang et al., J Alzheimers Dis. 2017; 58(1):1-15). Recently, the role of Interferon has been explored by researchers in Alzheimer's disease, although the oral route of delivery or basis of treatment on patients with similar gut biome has not been considered (https://www.brightfocus.org/alzheimers/grant/role-type-i-Interferon-alzheimers-disease).

The gut microbiome is a driver of the immune system's recognition of what is perceived as “self,” and what tissues or organ systems may be recognized as foreign. Without a gut microbiome containing all the concentrations and levels of microorganisms seen in a healthy/normal gut biome population, the “self” identification function of the gut microbiome may be lost. This leads to a potential immune cascade of attack against “self” if there is an external triggering antigen.

This inventor provides a new method of identification for Type 1 diabetes patients and those at risk for Type 1 diabetes with compositions to specifically target the Th17-initiated immune attack on the pancreas. This inventor posits that generation of an individual's ability to recognize “self” of one's own organs is based on a normal gut microbiome. One's immune system may mistake its own organ systems as foreign, based upon lack of microorganisms in number, concentration and appropriate ratios within the gut. Based upon the direct relationship between the gut biome on the formation of the immune barrier created Th17, a new therapeutic approach to diagnosis and treatment for disease is described.

Until now, diagnoses of diseases have predominately been made based on clinical symptoms. For example, 20% of patients diagnosed with rheumatoid arthritis (RA) are found to have no positive serological tests of an autoimmune disease, and the diagnosis is made solely based on clinical symptomatology and radiological findings. Rheumatoid factor (RF), despite the name, is not always specific for rheumatoid arthritis (RA) and is seen in other mixed connective tissue diseases, such as Sjogren's syndrome, and is found to be positive in many conditions.

When RA is suspected, testing may include anticyclic citrullinated peptide antibody (anti-CCP), yet negative levels do not exclude RA and positive levels do not guarantee a diagnosis as is the case with many autoimmune diseases. Some conditions can raise RF levels, such as: other autoimmune diseases, certain chronic infections, diabetes, bacterial endocarditis, cancer, normal aging, vaccinations and transfusions.

This inventor posits by the above illustrations, that current modalities for diagnosing diseases, may become obsolete. This inventor proposes a new methodology of diagnosis and treatment of diseases based upon the gut microbiome of the individual. Symptomatic patients without adequate treatment or when a severe disease may run in a family, an earlier diagnosis can be made with new potential treatment before the onset of disease by evaluating the patient's gut microbiome.

This modality of diagnosis may demonstrate that many more conditions are autoimmune than previously recognized. Many conditions with abnormal gut microbiomes, ranging from diseases that have commonly been considered autoimmune or inflammatory diseases, have been made based on clinical symptoms or antibody markers in the plasma, but the gut microbiome may also provide a unique ability to find patients at risk for a disease and treatment at the earliest stages of symptomatology and before an external antigen has even triggered an autoimmune response.

This inventor provides a new system of identification for Type 1 diabetes. This is possible even before a diagnosis, and treatment can be initiated to protect against an autoimmune attack against insulin-producing cells. This inventor has identified a Th17-inhibitor, Interferon alfa-2a, that was previously only used as a subcutaneous injection that was FDA approved in the US from 1986-2007 and was discontinued from use due to newer therapies for the diseases for which it was indicated including hepatitis C and Hairy Cell leukemia.

The mechanism of action of Interferon alfa-2a as an inhibitor of Th17, both in vitro and in vivo, was not known while it was approved for usage in the US. The mechanism of action of Interferon alfa-2a as an inhibitor of Th17 has now been shown by many groups (Moschen et al., 2008 Immunobiology. 213; 9-10: 79-787, Guo et al., J Clin Invest. 1; 118(5): 1680-1690, Zhang et al., PLOS ONE 6(12): e28432, Henry et al., J Immunol. 2010; 184(7): 3755-3767).

This inventor posits that an autoimmune disease may be considered any condition in which there is an aberrant gut biome. This inventor posits that a Th17-inhibitor, such as the delivery of Interferon alfa-2a directly to the gut may play a role in treatment of disease based upon the unique gut biome of the patient and specifically may play a role in many diseases, including Type 1 diabetes when used with a Reg peptide or EXTL3 receptor analog, or small molecule to the binding region on the REG gene receptor, or stimulatory antibody to the REG gene receptor binding region or beta cell, stem cell or islet transplant.

The Centers for Disease Control reported 168,000 annual hospitalizations in the US among Type 1 diabetes patients for life-threatening diabetic ketoacidosis and 245,000 emergency room visits per year for hypoglycemia (https://www.cdc.ov/diabetes/dfs/datastatistis/ational-diabetes-statistics-report.pdf). Despite new technologies, new forms of exogenous insulin, continuous glucose monitors and closed-loop insulin pumps, new functional islets with immune protection are required for the potential of insulin independence among patients with Type 1 diabetes.

The ability to utilize the gut microbiome to diagnose disease and predict disease and potentially intervene prior to clinical symptomatology is now possible utilizing the gut microbiome. Based on symptomatology of the disease state, the dosage may vary if a Th117-inhibitor is used for prevention versus treatment and likely varies by different disease states.

Many devastating diseases with imperfect or no treatment options have unique gut microbiomes, including Amyolateral Sclerosis (ALS) (Wright et al., Biol Res Nurs. 2018. 20(5):513-521, Autism Spectrum Disorders (Li et al, Front Cell Neurosci. 2017; 11: 120), Bipolar Disorder Evans et al., J Psychiatr Res. 2017; 87: 23-29) schizophrenia (Severance et al., Schizophr Res. 2016; 176(1): 23-35), frontotemporal lobar degeneration (Kim et al., 2017; 13(7) Supple. (P 1132-1133), and more recently the link between the gut microbiome and variety of specific brain disorders and diseases with each having a specific gut microbiome (Sherwin et al., Ann NY Academy of Sciences, 2018 (14201)1:5:5-25). Th17 may be etiologic to the exacerbations of these conditions.

Among patients with Rheumatoid Arthritis (RA), patients are depleted in the following bacteria: faecalibacterium, roseburia, subdoligranulum, ruminococcus and pseudobutyrivibrio, which are enriched in the gut microbiome in healthy individuals (Wu et al., Osteoarthritis and Cartilage. 2017:25; S 287-288). When triggered by an environmental antigen, such as in an infection, Th17 may view the joints as foreign tissue because the gut biome is insufficient in concentration of the normal gut flora that is required to “teach” Th17 to recognize the joint tissue as “self.” In Sjogren's syndrome, there is a reduction in bacteroides, parabacteroides, faecalibacterium, and prevotella (de Paiva et al., 2016. Scientific Reports 6:23561), which may also lead to a symptomatic autoimmune attack causing dry eyes and dry mouth.

There are more than two dozen diseases that occur at higher risk among patients with Type 1 diabetes and their families. For example, multiple sclerosis is three times more likely to occur among patients with diabetes and their families (Dorman et al., 2003 Diabetes Care; 26(11): 3192-3193). Amyolateral Sclerosis is seen in higher incidence in families in whom a member has Type 1 diabetes (Kioumourtzoglou et al., 2015. JAMA Neurol.; 72(8):905-11).

Parkinson's Disease has a 36% higher risk among patients with diabetes and is another condition with a uniquely aberrant gut microbiome for which a Th17-inhibitor may be used at the earliest onset of symptoms or as a preventative measure (Screenname et al., 2011 Diabetes Care 34:1102-1108). There is a significant decrease of prevotellaceae in the gut microbiome of patients with Parkinson's disease compared to healthy controls (Scheperjans et al., 2015. Mov Disord.; 30(3):350-8, Perez-Pardo et al., 2017. European Journal of Pharmacology. 817; 86-95).

The gut microbiome may be important for predicting disease, but environmental triggers play a major role in the actual attack initiated by Th17 (Marrosu et al, Lancet. 2002 April 27; 359(9316):1461-5). Those with first degree relatives with Type 1 diabetes who have two or more positive serological antibodies have a 100% risk of developing diabetes. This inventor posits that a Th17-inhibitor that has traditionally been used in the past as a subcutaneous therapy can be used orally to directly impact Th17 and Th17-initiated specific organ attack based upon the patient's gut microbiome. Using the gut biome to predict disease, offers the potential to treat many diseases associated with diabetes at the earliest signs of onset.

At present, the determination of one's gut microbiome can be performed for as little as $100 with methodology by which the stool is cultured for organisms using mass spectrometry and sequencing by polymerase chain reaction to determine the DNA of organisms. The testing is offered commercially and is often covered by insurance. Once the individual's mapping of the gut microbiome is performed, it is compared to the gut biomes of individuals that are suffering a disease. If there is similarity in the gut biomes, then it is determined that the individual is susceptible to suffering from that same disease.

Pilot studies, predominately in animal models from decades ago, have shown the safety and potential efficacy of the oral Th17-inhibitor, Interferon alfa 2-a, when given in dosages of less than 1/10,000 the subcutaneous dosage, but no dose ranging studies were conducted (Cummins. 2005 AJVR; 66(1):165-176). An earlier study in Type 1 diabetes in man using oral Interferon alfa-2a, which recruited patients in 2001, demonstrated the safety for usage of oral Interferon alfa-2a in children and adults, but efficacy in terms of insulin independence could not be seen and further studies were discontinued due to perceived lack of efficacy (Rother. Diabetes Care. 2009 July; 32(7): 1250-1255).

This inventor posits that signals were seen in the Type 1 study and there were no safety issues or side effects from oral Interferon alfa-2a, but the appropriate dosage was unclear with only patients receiving the lowest oral dosage (5000 IU per day) having signals of maintaining beta cell mass, while those on higher dosages did not. Other studies in animals and man using oral Interferon have used dosages orally as low as 100-200 IU daily (Gilger et al., J Interferon Cytokine Res. 1999; 19(8):901-5., Cummins et al., 2004, J Interferon Cytokine Res. (9):8, Lecciones et al., J Interferon Cytokine Res. 1998; 18(9):647-52, Shiozawa et al., J Interferon and Cytokine Research 18:225-262). This inventor posits that optimal dosing for prevention and treatment may be different and optimal dosages for treatment of different diseases may vary greatly.

Patients with Type 1 diabetes who demonstrate abnormal gut microbiomes, may be a candidate for oral Interferon alfa-2 with its mechanism of action as a Th17-inhibitor. This inventor posits that insulin independence is only possible for type 1 diabetes when both an islet neogenesis agent and an immune agent are used. More than 300 studies demonstrating success in reversing diabetes in Type 1 diabetes mouse models (NOD mice) have not been successful in man (Matthews et al., Clin Exp Immunol. 2010; 160(2): 176-184). Even when combinations of immune therapies have been given in humans with Type 1 diabetes, there has not been regeneration of insulin producing cells, thus Type 1 diabetes in man has been defined as both a disease of autoimmunity and lack of regeneration of insulin-producing cells even in an immune muted milieu.

It has been well documented in the literature, that patients and their first-degree relatives of patients with Type 1 diabetes have a higher incidence of many conditions, some autoimmune, and others have yet to be classified. If a patient has an abnormal gut biome and has a disease for which there is no effective treatment, a Th17-inhibitor, delivered directly to gut may be considered for many diseases that may or may not have been considered as autoimmune. This inventor posits that a new definition of autoimmune disease may potentially be based on the gut biome in the future. The optimal dosage for different disease states will need to be determined for each disease and based on symptomatology.

Researchers have long discussed the etiology of Alzheimer's Disease as an inflammatory disease and even have specifically discussed the role of the gut biome in the development of Alzheimer's Disease (Vogt et al., Scientific Reports, 2017 (7) Article 13537, Hu, China Life Sci. (2016) 59: 1006. https://doi.org/10.1007/s11427-016-5083-9 Jiang J Alzheimers Dis. 2017; 58(1):1-15). Recently, the role of Interferon has been explored by researchers in Alzheimer's disease, although the oral route of delivery or basis of treatment on patients with similar gut biome has not been considered (https://www.brightfocus.org/alzheimers/grant/role-type-i-Interferon-alzheimers-disease).

To date, no one in the art has considered the usage of the gut biome to diagnose and treat patients who demonstrate they are at risk for a Th17-initiated inflammatory or autoimmune attack. Nor has consideration been given to dosage and therapy based on disease state and risk for developing a disease based on the gut microbiome with development of dose ranging for usage of the Th17-inhibitor, Interferon alfa-2a given orally and directly to Th17 in the gut. When there is an aberrant gut biome, a Th17-inhibitor delivered orally may provide targeted effective therapy for treating and preventing disease that are likely get if Th17 is triggered by an outside antigen with initiation of an inflammatory or autoimmune attack, not only to the outside antigen, but to a specific organ system based on the individual's gut microbiome.

Reference will now be made in detail to various exemplary embodiments of the invention. The true scope of the invention is defined by the claims. Further, any features of any embodiment described herein are equally applicable to any other embodiment described herein or envisioned by one of ordinary skill in the art. The detailed description provided herein should not be construed to exclude features otherwise described with respect to another embodiment.

In one embodiment, the present invention provides a method of treating recent onset, existing Type 1 diabetes and Latent Autoimmune Diabetes of Adulthood (LADA) by initially evaluating the gut microbiome. If the gut biome is distinct for Type 1 diabetes, then, a Th17-inhibitor including, but not limited to the oral usage of Interferon alfa-2a or optimized forms of forms oral Interferon alfa 2a or other agents that inhibit Th17 may be used orally and in combination with an islet regeneration therapy, or islet neogenesis agents to include new therapies formed to the 20 amino-acid binding region on the REG gene receptor, or the oral Th17-inhibitor may also be used beta cell, stem cell or islet transplants within an encapsulation method for beta cells, islets or stem cells. The combination of an oral Th17-inhibitor used with a therapy that generates new endogenous insulin to patients with Type 1 diabetes or used with islets, beta cells, or stem cell transplanted into an individual may reduce or eliminate the need for exogenous insulin dependence in Type 1 diabetes patients.

Not wishing to be bound by theory, other conditions that demonstrate an aberrant gut microbiome for which there is inadequate or no treatment available may benefit from the use of a Th17-inhibitor given orally to prevent targeted organ attack based upon their gut biome. This novel approach to the usage of traditional immune therapies that has previously been given subcutaneously, intramuscularly, or intravenously, as in the prior art, also includes the new approach by this inventor of methods of usage of traditionally used immune therapies to be used in oral formulation, based on the gut microbiome determining if a Th17-inhibitor may be helpful with unique dosing required depending on the disease state.

The information on the gut microbiome extends to clinical trials where determination of the best dosage is based on the disease state reflected by the gut microbiome will be performed. Based on the degree of symptoms for a specific disease based on their gut microbiome, the dosage of therapy for the disease and prevention of disease will be determined.

### Examples

### Example 1

Patients with Type 1 diabetes with a gut biome consistent with Type 1 diabetes are randomized to one of 4 arms with 1) one study arm being given a placebo subcutaneous injection given daily with an oral dosage of placebo 2) an study arm receiving 400 mg daily of an optimized Reg peptide given subcutaneously daily with 300 IU of oral Interferon alfa-2a daily 3) an arm receiving 400 mg daily of an optimized Reg peptide given subcutaneously daily with 1000 IU of oral Interferon alfa-2a daily 4) an arm receiving 400 mg daily given subcutaneously of an optimized Reg peptide daily with 5000 IU of oral Interferon alfa-2a daily. Outcome measure include: Stimulated C-peptide Area Under the Curve (AUC), insulin requirements, glucose levels as measured by a continuous glucose monitoring system (CGMS) and hemoglobin AIC (AIC).

### Example 2

Patients with 2 or more antibodies including, but not limited to GAD65, insulin autoantibodies, ZNT8 antibodies and IA2 associated with a 100% change for the development of Type 1 diabetes, who have a gut microbiome consistent with that of patients with Type 1 diabetes are randomized in a dose-ranging study to one of 4 arms with the goal of the study to delay or prevent the onset of Type 1 diabetes. The treatment arms include: 1) one arm being a placebo arm 2) an arm receiving 300 IU of oral Interferon alfa 2a daily 3) an arm receiving 1000 IU of oral Interferon and 4) an arm receiving 5000 IU of oral Interferon alfa 2a daily. Outcome measure include: Stimulated C-peptide AUC, insulin requirements, glucose levels as measured by CGMS, AIC, percentage of patients who develop diabetes based upon Glucose Tolerance Testing and AIC.

### Example 3

In patients with a diagnosis of fatal Amyolateral Sclerosis (ALS) based upon clinical presentation for which there is no treatment. If the said patients were to have a gut microbiome consistent with those found among patients with a specific gut microbiome associated with ALS that is different from a healthy gut microbiome, patients will be given stepwise therapy with oral Interferon alfa-2a. Since there is currently limited treatments for this disease, patients symptomatic with a gut biome demonstrating the potential efficacy of using a Th17-inhibitor, patients will be dosed beginning at 300 IU orally of Interferon alfa 2a daily. If stabilization and improvement in symptoms are seen that patient will continue 300 IU of oral Interferon daily with oral Interferon. If symptoms progress or worsen after 2 weeks of therapy, the dosage will be increased to 1000 IU daily for 2 weeks, at which time if there is progression of the disease, the dosage will be increased to 5000 IU per day of oral Interferon. If stabilization and improvement in symptoms are seen that patient will continue the dosage 1000 IU of oral Interferon daily. The dosage will be increased every 2 weeks based on symptomatology or progression of the disease with the next 2-week increase to 10,000 IU orally per day followed by 20,000 IU for two weeks, followed by 30,000 IU orally daily. At any point that there is stabilization of disease, the said patient will remain on their current dose and the dose not escalated unless there is deterioration in symptoms.

### Example 4

A said patient with Alzheimer's Disease (AD) as identified by clinical markers and cognitive testing including Alzheimer's Disease Assessment Score, Cognitive Subscale ratings and Mini-Mental Status Exam and who has a specific gut microbiome that has been described among patients with progressive Alzheimer's Disease and has declining scores despite usage of available medications, will be considered for clinical trial with the oral Th17-inhibitor, oral Interferon alfa-2a. Patients in the study will be randomized to placebo, 100 IU of oral Interferon alfa-2a, 300 IU, 3000 IU, and 10,000 IU and followed at four-week intervals for evaluation for a total of 12 months. The goal of the study is to determine if there is an optimal dosage at which patients with Alzheimer's Disease with a gut microbiome consistent with Alzheimer's Disease have stabilization or significant lack of progression of their diseased as measured by Alzheimer's Disease Assessment Score, Cognitive Subscale ratings and Mini-Mental Status Exam.

### Example: 5

Said patient with diabetes is given an oral stimulatory antibody, binding site analog or small molecule generated to the 20 amino-acid binding site on the REG receptor, EXTL3, and acting as an islet neogenesis agent. The therapy will be evaluated with safety studies including single-dose dose escalation studies to determine the safest dosage tolerated. Based on findings patients, will be randomized to differing dosages of novel islet neogenesis agents generated from the 20 amino-acid binding region on the 920-amino acid Reg protein receptor. Based on safety data, patients with Type 1 diabetes will also be randomized to safety and efficacy trials using either an islet neogenesis agent with an oral Th17-inhibitor, including, but not limited to oral Interferon alfa 2a, based on their gut biome reflecting the gut microbiome of a patient with Type 1 diabetes. Type 2 patients and patients with PreDiabetes will not require an oral Th17-inhibitor but will only require an islet neogenesis agent.

1. A method of treating or preventing impaired glucose homeostasis in a subject comprising the steps of:
mapping the subject's gut microbiome;
comparing the subject's gut microbiome results with known mapped gut microbiomes of individuals suffering from particular forms of impaired glucose homeostasis;
determining the particular type of glucose impairment of the subject based on the subject's mapped gut microbiome, matching a known mapped gut microbiome of an individual suffering from a particular form of impaired glucose homeostasis;
using one or more islet or beta cell regeneration or replacement therapies; and
administering a T helper lymphocyte 17 (Th17)-inhibitor in an amount that is effective for protection of beta cells from immune-mediated destruction in the subject.

2. The method of claim 1, wherein the Th17-inhibitor is Interferon alfa-2a, PEGylated Interferon alfa-2a, or an optimized version of either.

3. The method of claim 2, wherein the Interferon alfa-2a or the PEGylated Interferon alfa-2a is administered orally to the subject.

4. The method of claim 3, wherein the Interferon alfa-2a or the PEGylated Interferon alfa-2a is administered to the subject at a dosage in the range of 100 to 50,000 IU.

5. The method of claim 1, wherein the one or more islet or beta cell regeneration or replacement therapies comprise one or more islet neogenesis or beta cell regeneration agents.

6. The method of claim 5, wherein the islet neogenesis or beta cell regeneration agent binding to the 20-amino acid binding region [SEQ ID: 3] of the 920-amino acid Reg receptor [SEQ: ID 4].

7. The method of claim 6, wherein islet neogenesis or beta cell regeneration agent is a binding site analog, a stimulatory antibody to the binding site, or a small molecule capable of binding to the Reg receptor binding region [SEQ ID: 3].

8. The method of claim 5, wherein the islet neogenesis or beta cell regeneration agent has been modified to increase its stability in plasma, increase its solubility, increase its protease resistance, reduce its immunogenicity, increase Tmax, and/or increase its bioavailability.

9. The method of claim 1, wherein the one or more islet or beta cell regeneration or replacement therapies comprise one or more of an islet transplant, beta cell transplant, or stem cell transplant.

10. The method of claim 1, wherein the one or more islet or beta cell regeneration or replacement therapies comprise one or more devices or modalities that house or encapsulate one or more of islets, stem cells or beta cells.

11. The method of claim 1, wherein conditions frequently seen in family members who have Type 1 diabetes who have progressive diseases which may have an autoimmune basis for which there may be no treatment available and methods above may stop the progression of such autoimmune diseases or conditions that is one or more of Amyotrophic Lateral Sclerosis (ALS), forms of Parkinson's disease, pulmonary fibrosis, pediatric autoimmune neurobiological disorders, Myasthenia gravis, Chronic inflammatory demyelinating polyneuropathy (CIDP), Multifocal motor neuropathy (MMN), POEMS syndrome (osteosclerotic myeloma: polyneuropathy, organomegaly, endocrinopathy, monoclonal protein, skin changes), anti-myelin associated glycoprotein (MAG)-related neuropathies, Combined Sensorimotor Neuropathy in Rheumatoid Arthritis, Juvenile Rheumatoid Arthritis, Frontotemporal Lobar Dementia and progressive neurologic conditions.

12. A method of treating or preventing one or more autoimmune diseases or conditions in a subject comprising:
mapping the subject's gut microbiome;
comparing the subject's gut microbiome results with known mapped gut microbiomes of other individuals suffering from a particular forms autoimmune disease or condition;
determining the particular type of autoimmune disease or condition of the subject based on the subject's mapped gut microbiome matching a known mapped gut microbiome of an individual suffering from a particular form of autoimmune disease or condition; and
administering a Th17-inhibitor at an amount that is effective for alleviating or preventing symptoms of the autoimmune disease or condition in the subject.

13. The method of claim 12, wherein the Th17-inhibitor is Interferon alfa-2a or PEGylated Interferon alfa-2a.

14. The method of claim 13, wherein the Interferon alfa-2a or the PEGylated Interferon alfa-2a is administered orally to the subject.

15. The method of claim 14, wherein the Interferon alfa-2a or the PEGylated Interferon alfa-2a is administered to the subject at a dosage in the range of 100 to 50,000 IU.

16. A method of diagnosing and treating one or more diseases that is based on a Th17-initiated inflammatory or autoimmune attack in a subject comprising:
mapping the subject's gut microbiome;
comparing the subject's gut microbiome results with known mapped gut microbiomes relating to aberrant gut microbiomes of individual's suffering symptoms of Th17-initiated inflammatory or autoimmune attacks; and
diagnosing the subject's particular type disease based on the subject's mapped gut microbiome matching a known mapped gut microbiome of an individual suffering from a particular form of Th17-initiated inflammatory or autoimmune attack.

17. The method of claim 16, further comprising the step of administering a Th17-inhibitor at an amount that is effective for alleviating or preventing symptoms of the Th17-initiated inflammatory or autoimmune attack in the subject.

18. The method of claim 17, wherein the Th17-inhibitor is Interferon alfa-2a or PEGylated Interferon alfa-2a.

19. The method of claim 18, wherein the Interferon alfa 2-a or the PEGylated Interferon alfa-2a is administered orally to the subject.

20. The method of claim 19, wherein the Interferon alfa 2-a or the PEGylated Interferon alfa-2a is administered to the subject at a dosage in the range of 100 to 50,000 IU.
